(19) United States
(12) Patent Application Publication
(10) Pub. No.: US 2004/0029097 A1
(43) Pub. Date: Feb. 12, 2004

(54) Addition of glycolysis inhibitor to a pathogen reduction and storage solution

(75) Inventors: Laura Goodrich, Lakewood, CO (US); Raymond P. Goodrich, Lakewood, CO (US)

Correspondence Address: GAMBRO, INC, PATENT DEPARTMENT, 10810 W COLLINS AVE, LAKEWOOD, CO, 80215, US

(73) Assignee: Gambro, Inc., Lakewood, CO

(21) Appl. No.: 10/417,925

(22) Filed: Apr. 16, 2003

Related U.S. Application Data
(63) Continuation of application No. 10/355,681, filed on Jan. 31, 2003.
(60) Provisional application No. 60/373,198, filed on Apr. 16, 2002.

(51) Int. Cl. A01N 1/02; A61K 31/70

(52) U.S. Cl. 435/2; 514/23; 514/250

(57) ABSTRACT

This invention relates to the addition of glycolytic inhibitors to solutions used in the pathogen reduction and subsequent storage of platelets. More particularly, the invention relates to the addition of 2-deoxy-D-glucose and sodium succinate to a platelet pathogen reduction and storage solution.

### CROSS REFERENCE To RELATED APPLICATIONS

This application claims priority to U.S. Provisional patent application Ser. No. 60/373,198 filed Apr. 16, 2002, and U.S. patent application Ser. No. 10/355681 filed Feb. 1, 2003.

### BACKGROUND OF THE INVENTION

The breakdown of glucose to provide energy to cells is an important mechanism in cellular metabolism. This mechanism, known as glycolysis, produces ATP (adenosine triphosphate) in the absence of oxygen. The production of ATP is essential for cellular energy metabolism.

In glycolysis, one glucose molecule having six carbon atoms is converted into two molecules of pyruvate, each having three carbon atoms. This conversion involves a sequence of nine enzymatic steps that create phosphate-containing intermediates. The cell hydrolyzes two molecules of ATP to drive the early steps, but produces four molecules of ATP in the later steps.

For most animal cells, glycolysis is merely the first stage in the breakdown of sugar into cellular energy, since the pyruvic acid that is formed at the last step quickly enters the cell's mitochondria to be completely oxidized to CO and H₂O in the citric acid cycle. In this final step of the cycle, intermediate energy carrying molecules NADH (nicotinamide adenine dinucleotide) and FADH₂ (flavin adenine dinucleotide) transfer electrons they have gained from the oxidation of glucose in the glycolysis pathway to molecular oxygen. These electrons are transferred as an electron plus a proton (H+) down a long chain of carrier molecules called the electron transport chain. At the end of this chain, one molecule of oxygen picks up four electrons and four protons (H+) to form two molecules of water. This electron transport occurs on the inner membrane of the mitochondria.

Since ATP is essential to continued cell function, when aerobic metabolism is slowed or prevented by lack of oxygen, anaerobic pathways for producing ATP are stimulated and become critical for maintaining cell viability. Here, instead of being degraded in the mitochondria, the pyruvate molecules stay in the cytosol of the cell and can be converted into ethanol and CO₂ (as in yeast) or into lactate (as in muscle).

In the case of organisms which are anaerobic (those that do not use molecular oxygen) and for tissues like skeletal muscle that can function under anaerobic conditions, glycolysis can become a major source of the cell's ATP. This also occurs if the mitochondria of the cell is damaged in some way, thereby preventing the cell from entering the citric acid cycle.

Lactate accumulation in cells undergoing glycolysis causes an increased concentration of hydrogen ions (a decreased pH). If cells undergoing glycolysis are being stored, such a drop in pH might contribute to a decrease in cell quality during cell storage. In the case of platelets, pH drop might cause an increase in platelet activation during storage.

Factors which might cause cells to enter glycolysis and thereby accumulate lactic acid or lactate may be events which occur internally in a body such as strokes or infarctions, or may be caused by external events such as treatment of the cells after removal from a body. One example of an external treatment which might cause cells to accumulate lactate is a procedure to inactivate any pathogens which might be contained in or around cells to be transfused into a recipient. Currently used methods to sterilize pathogenic contaminants which may be present in blood cells (including red blood cells, white blood cells and platelets) may cause damage to the mitochondria of the cells being treated, for instance ultraviolet light has been shown to damage mitochondria. If this occurs, the cells can only make ATP through the glycolysis pathway, causing a buildup of lactic acid in the cell, and a subsequent drop in pH during storage.

One method used to sterilize blood and blood components requires the use of photosensitizers, compounds which absorb light of a defined wavelength and transfer the absorbed energy to an energy acceptor. For example, European Patent application 196,515 published Oct. 8, 1986, suggests the use of non-endogenous photosensitizers such as porphyrins, psoralens, acridine, toluidines, flavine (acriflavine hydrochloride), phenothiazine derivatives, and dyes such as neutral red and methylene blue, as blood additives. Protoporphyrin, which occurs naturally within the body, can be metabolized to form a photosensitizer; however, its usefulness is limited in that it degrades desired biological activities of proteins. Chlorpromazine is also exemplified as one such photosensitizer; however its usefulness is limited by the fact that it should be removed from any fluid administered to a patient after the decontamination procedure because it has a sedative effect.

Goodrich, R. P., et al. (1997), “The Design and Development of Selective, Photoactivated Drugs for Sterilization of Blood Products,” Drugs of the Future 22:159-171 provides a review of some photosensitizers including psoralens, and some of the issues of importance in choosing photosensitizers for decontamination of blood products. The use of texaphyrins for DNA photocleavage is described in U.S. Pat. No. 5,607,924 issued Mar. 4, 1997 and 5,714,328 issued Feb. 3, 1998 to Magda et al. The use of sapphyrins for viral deactivation is described in U.S. Pat. No. 5,041,078 issued Aug. 20, 1991 to Matthews, et al. Inactivation of extracellular enveloped viruses in blood and blood components by Phenthiazin-5-ium dyes plus light is described in U.S. Pat. No. 5,545,516 issued Aug. 13, 1996 to Wagner. The use of porphyrins, hematoporphyrins, and merocyanine dyes as photosensitizing agents for eradicating infectious contaminants such as viruses and protozoa from body tissues such as body fluids is disclosed in U.S. Pat. No. 4,915,683 issued Apr. 10, 1990 and related U.S. Pat. No. 5,304,113 issued Apr. 19, 1994 to Sieber et al.

The mechanism of action of psoralens is described as involving preferential binding to domains in lipid bilayers, e.g. on enveloped viruses and some virus-infected cells. Photoexcitation of membrane-bound agent molecules leads to the formation of reactive oxygen species such as singlet oxygen which causes lipid peroxidation. A problem with the use of psoralens is that they attack cell membranes of desirable components of fluids to be decontaminated, such as red blood cells, and the singlet oxygen produced during the reaction also attacks desired protein components of fluids being treated.

U.S. Pat. No. 4,727,027 issued Feb. 23, 1988 to Wiesehahn, G. P., et al. discloses the use of furocoumarins including psoralen and derivatives for decontamination of blood and blood products, but teaches that steps must be taken to reduce the availability of dissolved oxygen and other reactive species in order to inhibit denaturation of biologically active proteins. Photoinactivation of viral and bacterial blood contaminants using halogenated coumarins is described in U.S. Pat. No. 5,516,629 issued May 14, 1996 to Park, et al. U.S. Pat. No. 5,587,490 issued Dec. 24, 1996 to Goodrich Jr., R. P., et al. and U.S. Pat. No. 5,418,130 to Platz, et al. disclose the use of substituted psoralens for inactivation of viral and bacterial blood contaminants. The latter patent also teaches the necessity of controlling free radical damage to other blood components. U.S. Pat. No. 5,654,443 issued Aug. 5, 1997 to Wollowitz et al. teaches new psoralen compositions used for photodecontamination of blood.

It is known in the art to use photosensitizers as a component of a solution for the pathogen reduction of blood. For example, U.S. Pat. No. 5,709,991 to Lin et al. teaches the use of psoralen for photodecontamination of platelet preparations and removal of photolysed psoralen afterward. U.S. Pat. No. 5,459,030 also issued to Lin teaches a platelet storage medium containing 8-methoxypsoralen for use in a pathogen reduction process. U.S. Pat. Nos. 5,712,085, 5,908,742, 5,955,256, 5,965,349, 6,017,691 and 6,251,580 all disclose solutions for use in the pathogen reduction of blood which all include psoralen or psoralen derivatives as the photosensitizer. None of these disclosed solutions suggests the addition of glycolytic inhibitors to help in the pathogen reduction and subsequent storage of the pathogen reduced blood and/or blood products.

U.S. Pat. No. 5,120,649 issued June 9, 1992 and related U.S. Pat. No. 5,232,844 issued Aug. 3, 1993 to Horowitz, et al., disclose the need for the use of “quenchers” in combination with photosensitizers which bind to lipid membranes, and U.S. Pat. No. 5,360,734 issued Nov. 1, 1994 to Chapman et al. also addresses this problem of prevention of damage to other blood components.

Photosensitizers which attack nucleic acids are known to the art. U.S. Pat. No. 5,342,752 issued Aug. 30, 1994 to Platz et al. discloses the use of compounds based on acridine dyes to reduce parasitic contamination in red blood cells, platelets, and blood plasma protein fractions. These materials, although of fairly low toxicity, do have some toxicity especially to red blood cells. This patent fails to disclose an apparatus for decontaminating blood on a flow-through basis. U.S. Pat. No. 5,798,238 to Goodrich, Jr., et al., discloses the use of quinolone and quinolone compounds for inactivation of viral and bacterial contaminants.

Binding of DNA with photoactive agents has been utilized in processes to reduce lymphocytic populations in blood as taught in U.S. Pat. No. 4,612,007 issued Sep. 16, 1986 and related U.S. Pat. No. 4,683,889 issued Aug. 4, 1987 to Edelson.

The vitamin riboflavin (7,8-dimethyl-10-ribityl isoalloxazine) has also been reported to attack nucleic acids. U.S. Pat. Nos. 6,258,577 and 6,277,337 issued to Goodrich et al. disclose the use of riboflavin (an endogenous photosensitizer) and light to inactivate microorganisms which may be contained in blood or blood products. U.S. Pat. No. 6,268,120 to Platz et al. discloses riboflavin derivatives which may also be used to inactivate microorganisms.

All publications referred to herein are hereby incorporated by reference to the extent not inconsistent herewith.

The present invention is directed to a solution containing glycolysis inhibitors for irradiating and storing platelets to help maintain the quality of platelets during a pathogen reduction process, as well as for long term storage of the pathogen reduced platelet product.

### SUMMARY OF THE INVENTION

This invention relates to the addition of glycolytic inhibitors to solutions containing platelets which have been or which will be subjected to a pathogen reduction process, in order to help maintain the quality of the platelets during the process as well as afterwards during storage. More particularly, the invention relates to the addition of 2-deoxy-D-glucose and sodium succinate to a platelet pathogen reduction and/or storage solution. In a further embodiment, 2-deoxy-D-glucose and sodium succinate is added to a solution containing platelets in an amount between about 1-10 mM.

### BRIEF DESCRIPTION OF THE FIGURES

FIG. 1 is a graph showing the production of lactate by pathogen reduced platelets over five days of storage.

FIG. 2 is a graph showing the consumption of glucose by pathogen reduced platelets over five days of storage.

FIG. 3 is a graph showing changes in pH by pathogen reduced platelets over five days of storage.

FIG. 4 is a graph showing changes in ATP production by pathogen reduced platelets over five days of storage.

FIG. 5 shows an embodiment of this invention using a bag to contain the fluid being treated with the photosensitizer and glycolytic inhibitors and a shaker table to agitate the fluid while exposing to photoradiation from a light source.

### DETAILED DESCRIPTION OF THE INVENTION

A solution is provided for maintaining platelet viability either before, during or after a pathogen reduction process.

The photosensitizers useful in this invention include any photosensitizers known to the art to be useful for inactivating microorganisms or other infectious particles. A “photosensitizer” is defined as any compound which absorbs radiation at one or more defined wavelengths and subsequently utilizes the absorbed energy to carry out a chemical process. Examples of such photosensitizers include porphyrins, psoralens, dyes such as neutral red, methylene blue, acridine, toluidines, flavine (acriflavine hydrochloride) and phenothiazine derivatives, coumarins, quinolones, quinones, and anthroquinones. Photosensitizers of this invention may include compounds which preferentially adsorb to nucleic acids, thus focusing their photodynamic effect upon microorganisms and viruses with little or no effect upon accompanying cells or proteins. Other photosensitizers are also useful in this invention, such as those using singlet oxygen-dependent mechanisms.

Most preferred are endogenous photosensitizers. The term “endogenous” means naturally found in a human or mammalian body, either as a result of synthesis by the body or because of ingestion as an essential foodstuff (e.g. vitamins) or formation of metabolites and/or byproducts in vivo. Examples of such endogenous photosensitizers are alloxazines such as 7,8-dimethyl-10-ribityl isoalloxazine (riboflavin), 7,8,10-trimethylisoalloxazine (lumiflavin), 7,8-dimethylalloxazine (lumichrome), isoalloxazine-adenine dinucleotide (flavine adenine dinucleotide [FAD]), alloxazine mononucleotide (also known as flavine mononucleotide [FMN] and riboflavine-5-phosphate), vitamin Ks, vitamin L, their metabolites and precursors, and napththoquinones, naphthalenes, naphthols and their derivatives having planar molecular conformations. The term “alloxazine” includes isoalloxazines. Endogenously-based derivative photosensitizers include synthetically derived analogs and homologs of endogenous photosensitizers which may have or lack lower (1-5) alkyl or halogen substituents of the photosensitizers from which they are derived, and which preserve the function and substantial non-toxicity thereof. When endogenous photosensitizers are used, particularly when such photosensitizers are not inherently toxic or do not yield toxic photoproducts after photoradiation, no removal or purification step is required after decontamination, and the treated product can be directly administered to a patient by any methods known in the art. Preferred endogenous photosensitizers are:

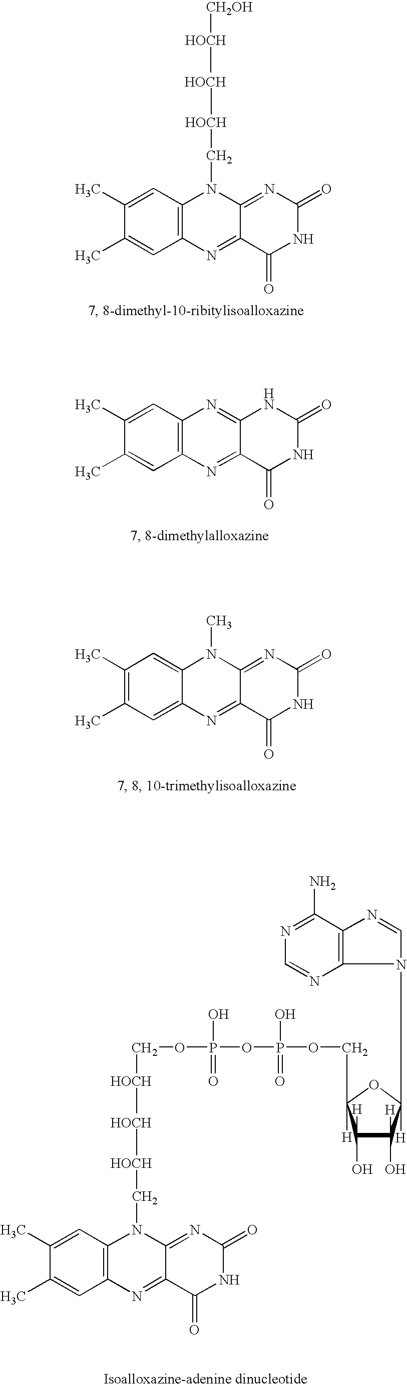

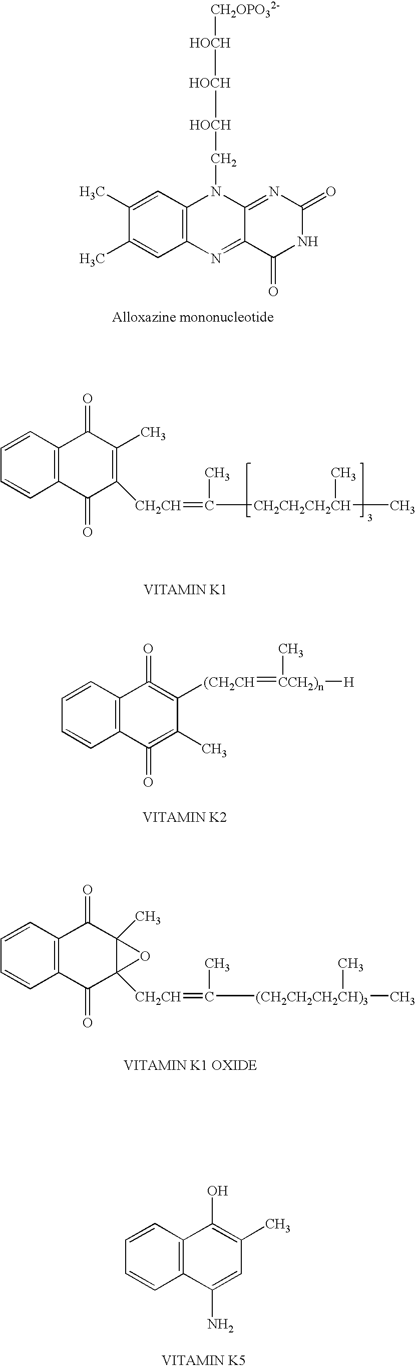

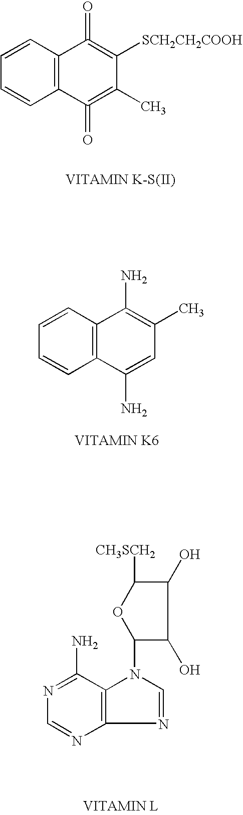

The method of this invention requires mixing the selected photosensitizer and the glycolytic or glycolysis inhibitors with the fluid containing platelets to be pathogen reduced. Glycolytic inhibitors are added to the fluid containing platelets at a concentration of between about 1 mM to 10 mM. The glycolytic inhibitors may be added to the fluid containing platelets either before the addition of the photosensitizer or after addition of the photosensitzer. The inhibitors may also be added after the platelets and photosensitizer have been exposed to photoradiation.

The amount of photosensitizer to be mixed with the platelets will be an amount sufficient to adequately inactivate the reproductive ability of a pathogen. For 7,8-dimethyl- 10-ribityl isoalloxazine a concentration range between about 1 μM and about 160 μM is used, preferably about 50 μM. The photosensitizer may be added directly to the platelets to be pathogen reduced, or may be added to the platelets to be decontaminated in a pre-mixed aqueous solution, e.g., in water, storage buffer or suspension solution.

In one embodiment, the photosensitizer and/or glycolysis inhibitors are added to a fluid which is used to suspend the blood components to be pathogen reduced. In another embodiment, the photosensitizer and/or glycolysis inhibitors may be added directly to the blood components to be inactivated. In the description above it is further understood that the photosensitizer and glycolysis inhibitor can each be separately added to the platelets to be pathogen reduced.

In one embodiment, the platelets to which a photosensitizer and glycolytic inhibitors have been added is flowed past a photoradiation source, and the flow of the material generally provides sufficient turbulence to distribute the photosensitizer and glycolytic inhibitors throughout the fluid to be pathogen reduced. A mixing step may optionally be added.

In another embodiment, the fluid, photosensitizer and glycolysis inhibitor are placed in a photopermeable container and irradiated in batch mode, preferably while agitating the container to fully distribute the photosensitizer and glycolytic inhibitors throughout the fluid and expose all the fluid to the radiation. In this embodiment, the photopermeable container is preferably a blood bag made of transparent or semitransparent plastic, and the agitating means is preferably a mechanism for shaking the container in multiple planes.

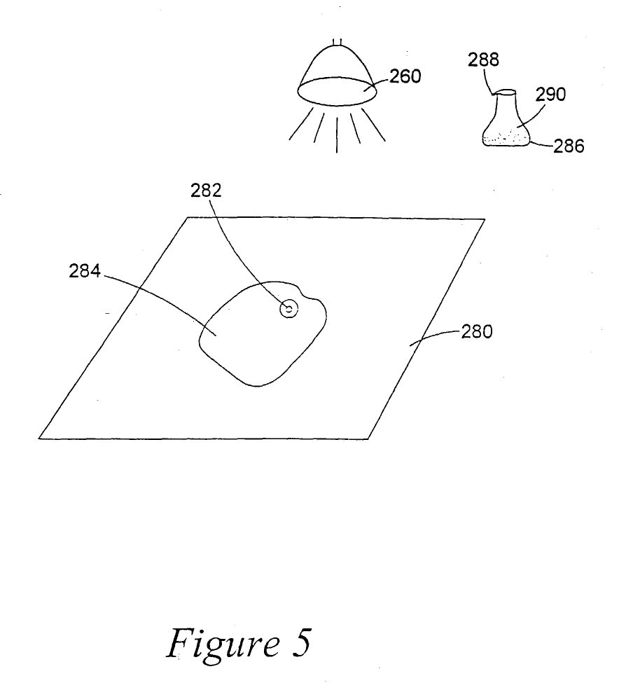

FIG. 5 depicts an embodiment of this invention in which fluid to be decontaminated is placed in a bag 284 equipped with an inlet port 282, through which photosensitizer and glycolysis inhibitors 290 may be added from flask 286 via pour spout 288. Shaker table 280 is activated to agitate the bag 284 to mix the fluid to be decontaminated, the photosensitizer and the glycolysis inhibitors together while photoradiation source 260 is activated to irradiate the fluid and photosensitizer in bag 284. The photosensitizer and/or glycolysis inhibitors may be added to the container in powdered or liquid form, or alternatively, the bag 284 can be provided prepackaged to contain photosensitizer and glycolysis inhibitors and the platelets may thereafter be added to the bag. The glycolysis inhibitors may also be added to bag 284 through a sterile barrier filter (not shown) connected to inlet port 282.

The fluid containing the photosensitizer and glycolytic inhibitors is exposed to photoradiation of the appropriate wavelength to activate the photosensitizer, using an amount of photoradiation sufficient to activate the photosensitizer as described above, but less than that which would cause severe damage to the platelets being pathogen reduced. The addition of glycolytic inhibitors as described may help platelets maintain their viability after exposure to a photosensitizer and light.

The photoradiation source may be a simple lamp or may consist of multiple lamps radiating at differing wavelengths. The photoradiation source should be capable of delivering from about 1 J/cm² to at least about 120 J/cm^{2.}

The wavelength emitted from the photoradiation source will depend on the type of photosensitizer selected and the type of blood component being pathogen reduced. Wavelengths in the ultraviolet to visible range are useful in this invention. The photoradiation source or sources may provide light in the visible range, light in the ultraviolet range, or may be a mixture of light in both the visible and ultraviolet ranges. For platelets, the light source may provide light of about 270 nm to about 700 nm, and more preferably about 308 nm to about 320 nm.

Decontamination systems as described above may be designed as stand-alone units or may be easily incorporated into existing apparatuses known to the art for reducing pathogens in blood or blood components. The process is further described and is incorporated in its entirety to the amount not inconsistent in U.S. Pat. Nos. 6,277,337 and 6,258,577.

Quenchers may also be added to the fluid to make the process more efficient and selective. Such quenchers include antioxidants or other agents to prevent damage to desired fluid components or to improve the rate of inactivation of pathogens and are exemplified by adenine, histidine, cysteine, tyrosine, tryptophan, ascorbate, N-acetyl-L-cysteine, propyl gallate, glutathione, mercaptopropionylglycine, dithiothreotol, nicotinamide, BHT, BHA, lysine, serine, methionine, glucose, mannitol, vitamin E, trolox, alpha-tocopheral acetate and various derivatives, glycerol, and mixtures thereof.

It has been observed that one possible side effect of a pathogen reduction process is that when platelets are subjected to UV light, the mitochondria of the platelets have a greater chance of suffering at least some damage. If mitochondrial function is suppressed by UV light, platelets are unable to create ATP (energy) through aerobic respiration. If platelets are unable to create energy through aerobic respiration, they will create energy via the glycolysis pathway. As described above, one metabolite produced by the glycolysis pathway is lactic acid. Lactic acid buildup within cells causes the pH of the solution to drop. Such a drop in pH may cause decreased cell quality during storage, and platelet activation.

One way to prevent this pH drop and subsequent drop in cell quality would be to prevent the buildup of lactic acid. This may be done by using an agent or combination of agents which block or slow glycolysis.

2-deoxy-D-glucose is one such glycolytic inhibitor agent which slows the rate of glycolysis by competing with glucose for enzymes utilized in the glycolysis pathway. In the glycolysis pathway, 2-deoxy-D-glucose is phosohorylated by the same enzymes which phosphorylate glucose, but at a slower rate than that of glucose phosphorylation. Such competitive binding slows the rate of glucose breakdown by the cell and consequently slows the rate of lactic acid production.

Sodium succinate (C₄H₄Na₂O₄.6H₂O) is another glycolytic inhibitor agent which is thought to slow the rate of glycolysis. Sodium succinate is a metabolite used by cells to enhance uptake of H⁺ ions in the electron transport chain by the mitochondria. By enhancing the production of ATP through the citric acid pathway, the cell is driven to produce ATP through the citric acid pathway, not through the glycolysis pathway, and consequently a buildup of lactic acid is prevented.

It has been suggested that 2-deoxy-D-glucose used in combination with sodium succinate may help a cell undergoing a pathogen reduction procedure or which has previously undergone a pathogen reduction procedure to maintain energy (ATP) production. 2-deoxy-D-glucose may help to slow the glycolysis pathway, while sodium succinate may help the cell produce ATP through the citric acid cycle. By slowing the glycolysis pathway and by increasing ATP production through the citric acid cycle, production of lactic acid by stored pathogen reduced platelets may be substantially reduced.

In one embodiment, 2-deoxy-D-glucose and sodium succinate are added to a fluid containing a platelet suspension before the platelets are subjected to a pathogen reduction procedure. In a preferred embodiment, 2-deoxy-D-glucose is added to the fluid at a concentration of 1 mM to 10 mM, and sodium succinate is added to the fluid at a concentration of 1 mM to 10 mM. These agents may be added alone or in combination. The presence of these agents should slow the production of lactic acid during storage, allowing maintenance of pH, and consequently, cell quality during prolonged storage should be better maintained. The platelets could then be pathogen reduced according the pathogen reduction procedure described above.

In an alternative embodiment, 2-deoxy-D-glucose and sodium succinate may be added to platelets after a pathogen reduction procedure, to aid in storage of the pathogen reduced platelets. The following aqueous blood component additive solutions are some examples not meant to be limiting, of readily available commercial platelet additive solutions which may be used with the present invention.

### EXAMPLE 1

This example compares solvents which are novel blood component additive solutions for addition to platelets separated from whole blood. Six commercially available solutions were used: PAS II, PSMI-pH, PlasmaLyte A, SetoSol, PAS III, and PAS. To each known solution was added an effective amount of an endogenous photosensitizer, 7,8-dimethyl-10-ribityl isoalloxazine and a glycolytic inhibitor either alone or in combination with another glycolytic inhibitor. The photosensitizer may be present in the various solutions at any desired concentration from about 1 μM up to the solubility of the photosensitizer in the fluid, or dry medium. For 7,8-dimethyl-10-ribityl isoalloxazine a final concentration in a range between about 1 μM and about 160 μM is preferred, preferably about 50 μM. The glycolytic inhibitors may be present in the various solutions in a final concentration in an amount between 2 mM to 10 mM. For 2-deoxy-D-glucose a final concentration of about 10 mM is preferred. For sodium succinate a final concentration of about 2 mM is preferred. The final working composition of each solution is shown in Table 1a below, and varies in the amount of blood component additives present. The blood additive components may be in a physiological solution, as well as a dry medium adapted to be mixed with a solvent, including tablet, pill or capsule form.

TABLE 1a

| | Platelet Storage Solution | | | | | |
|---|---|---|---|---|---|---|
| Blood Component | PSS | PSS | PSS | PSS | PSS | PSS |
| Additive | 1 | 2 | 3 | 4 | 5 | 6 |
| KCl (mM) | | 5.0 | 5.0 | 5.0 | 5.1 | |
| CaCl₂ (mM) | | | | | 1.7 | |
| MgCl₂ (mM) | | | 3.0 | 3.0 | | |
| MgSO₄ (mM) | | | | | 0.8 | |
| sodium citrate (mM) | 10.0 | 23.0 | 23.0 | 17.0 | 15.2 | 12.3 |
| citric acid (mM) | | | | | 2.7 | |
| NaHCO₃ (mM) | | | | | 35.0 | |
| Na₂HPO₄ (mM) | | 25.0 | | 25.0 | 2.1 | 28.0 |
| sodium acetate (mM) | 30.0 | | 27.0 | 23.0 | | 42.0 |
| sodium gluconate (mM) | | | 23.0 | | | |
| glucose (mM) | | | | 23.5 | 38.5 | |
| maltose (mM) | | | | 28.8 | | |
| 7,8-dimethyl 10-ribityl | 50.0 | 50.0 | 50.0 | 50.0 | 50.0 | 50.0 |
| isoalloxazine (μM) | | | | | | |
| 2-deoxy-D-glucose | 10.0 | 10.0 | 10.0 | 10.0 | 10.0 | 10.0 |
| (mM) | | | | | | |
| sodium succinate (mM) | 2.0 | 2.0 | 2.0 | 2.0 | 2.0 | 2.0 |

As shown in Table 1a, the platelet storage solution PSS 1 comprises a physiological saline solution, tri-sodium citrate at a concentration of approximately about 10 mM, sodium acetate at a concentration of approximately about 30 mM, 7, 8-dimethyl-10-ribityl isoalloxazine at a concentration of about 50 μM, 2-deoxy-D-glucose at a concentration of about 10 mM and sodium succinate at concentration of about 2 mM.

The platelet storage solution PSS 2 comprises a physiological saline solution, potassium chloride at a concentration of approximately about 5 mM, tri-sodium citrate at a concentration of approximately about 23 mM, a mixture of monosodium phosphate and dibasic sodium phosphate at a concentration of approximately about 25 mM, 7,8-dimethyl-10-ribityl isoalloxazine at a concentration of about 50 μM, 2-deoxy-D-glucose at a concentration of about 10 mM and sodium succinate at concentration of about 2 mM.

Platelet storage solution PSS 3 comprises a physiological saline solution, potassium chloride at a concentration of approximately about 5 mM, magnesium chloride at a concentration of approximately about 3 mM, tri-sodium citrate at a concentration of approximately about 23 mM, sodium acetate at a concentration of approximately about 27 mM, sodium gluconate at a concentration of approximately about 23 mM, 7,8-dimethyl-10-ribityl isoalloxazine at a concentration of about 50 μM, 2-deoxy-D-glucose at a concentration of about 10 mM and sodium succinate at concentration of about 2 mM.

The platelet storage solution PSS 4 comprises a physiological saline solution, potassium chloride at a concentration of approximately about 5 mM, magnesium chloride at a concentration of approximately about 3 mM, tri-sodium citrate at a concentration of approximately about 17 mM, sodium phosphate at a concentration of approximately about 25 mM, sodium acetate at a concentration of approximately about 23 mM, glucose at a concentration of approximately about 23.5 mM, maltose at a concentration of approximately about 28.8 mM, 7,8-dimethyl-10-ribityl isoalloxazine at a concentration of about 50 μM, 2-deoxy-D-glucose at a concentration of about 10 mM and sodium succinate at concentration of about 2 mM.

Platelet storage solution PSS 5 comprises a physiological saline solution, potassium chloride at a concentration of approximately about 5.1 mM, calcium chloride at a concentration of approximately about 1.7 mM, magnesium sulfate at a concentration of approximately about 0.8 mM, tri-sodium citrate at a concentration of approximately about 15.2 mM, citric acid at a concentration of approximately about 2.7 mM, sodium bicarbonate at a concentration of approximately about 35 mM, sodium phosphate at a concentration of approximately about 2.1 mM, glucose at a concentration of approximately about 38.5 mM, 7,8-dimethyl-10-ribityl isoalloxazine at a concentration of about 10 μM, 2-deoxy-D-glucose at a concentration of about 10 mM and sodium succinate at concentration of about 2 mM.

As shown above in Table 1a, the platelet storage solution PSS 6 comprises a physiological saline solution, tri-sodium citrate at a concentration of approximately about 12.3 mM, sodium phosphate at a concentration of approximately about 28 mM, sodium acetate at a concentration of approximately about 42 mM, 7,8-dimethyl-10-ribityl isoalloxazine at a concentration of about 50 μM and 2-deoxy-D-glucose at a concentration of about 10 mM and sodium succinate at concentration of about 2 mM.

The physiologic saline solution may be replaced with a solvent comprising water and an effective amount of sodium chloride. 2-deoxy-D-glucose and sodium succinate may also be added to a solution containing saline or water and an effective amount of 7,8-dimethyl-10-ribityl isoalloxazine.

The platelet additive solution could comprise other additive solutions including an effective amount of 7,8-dimethyl-10-ribityl isoalloxazine and glycolytic inhibitors in a liquid, pill or dry medium form. PSS 7, PSS 8 and PSS 9 are further examples of such blood additive solutions set forth in Table 1b below. A quencher or combination of quenchers such as any disclosed above may also be added.

TABLE 1b

| | Platelet Storage Solution | | |
|---|---|---|---|
| Blood Component Additive | PSS 7 | PSS 8 | PSS 9 |
| NaCl (mM) | 115.0 | 78.3 | 68.5 |
| potassium cloride (mM) | | 5.7 | 5.0 |
| MgCl₂ (mM) | | 1.7 | 1.5 |
| sodium citrate (mM) | 10.0 | | |
| sodium phosphate (monobasic) | 6.2 | 5.4 | 8.5 |
| sodium phosphate (dibasic) | 19.8 | 24.6 | 21.5 |
| sodium acetate (mM) | 30.0 | 34.3 | 30.0 |
| 7,8-dimethyl 10-ribityl isoalloxazine (μM) | 50.0 | variable | 50.0 |
| 2-deoxy-D-glucose (mM) | 10.0 | 10.0 | 10.0 |
| sodium succinate (mM) | 2.0 | 2.0 | 2.0 |

As described in Table 1b, PSS 7 was prepared in RODI water and sodium chloride at a concentration of approximately 115 mM, sodium citrate at a concentration of approximately 10.0 mM, sodium phosphate (monobasic) at a concentration of approximately 6.2 mM, sodium phosphate (dibasic) at a concentration of approximately 19.8 mM, sodium acetate at a concentration of approximately 30.0 mM, 7,8-dimethyl 10-ribityl isoalloxazine at a concentration of approximately 50.0 μM, 2-deoxy-D-glucose at a concentration of approximately 10 mM and sodium succinate at concentration of about 2 mM. It has a pH of 7.2.

PSS 8 was prepared in RODI water and comprises and sodium chloride at a concentration of approximately 78.3 mM, potassium chloride at a concentration of approximately 5.7 mM, magnesium chloride at a concentration of approximately 1.7 mM, sodium phosphate (monobasic) at a concentration of approximately 5.4 mM, sodium phosphate (dibasic) at a concentration of approximately 24.6 mM, sodium acetate at a concentration of approximately 34.3 mM, a variable concentration of 7,8-dimethyl 10-ribityl isoalloxazine, 2-deoxy-D-glucose at a concentration of approximately 10 mM and sodium succinate at concentration of about 2 mM. It has a pH of 7.4, and an osmolarity of 297 mmol/kg.

PSS 9 was prepared in RODI water and comprises and sodium chloride at a concentration of approximately 68.5 mM, potassium chloride at a concentration of approximately 5.0 mM, magnesium chloride at a concentration of approximately 1.5 mM, sodium phosphate (monobasic) at a concentration of approximately 8.5 mM, sodium phosphate (dibasic) at a concentration of approximately 21.5 mM, sodium acetate at a concentration of approximately 30.0 mM, 7,8-dimethyl 10-ribityl isoalloxazine at a concentration of approximately 50.0 μM, 2-deoxy-D-glucose at a concentration of approximately 10 mM and sodium succinate at concentration of about 2 mM. It has a pH of 7.2, and an osmolarity of 305 mmol/kg.

It is understood that in PSS 7, PSS 8 and PSS 9 the RODI water and sodium chloride can be replaced with a saline solution.

It is also contemplated that a platelet additive solution in accordance with this invention can comprise 7,8-dimethyl-10-ribityl isolloxazine, glycolysis inhibitors and a quencher or combination of quenchers as described above.

### EXAMPLE 2

The quality of platelets stored for five days after being subjected to a pathogen reduction procedure may be measured using standard measures of platelet quality known in the art.

The following figures graph the quality of pathogen reduced platelets over a five day storage period. Platelets were separated from whole blood and collected using a blood collection device such as the COBE Spectra™ or TRIMA® apheresis systems available from Gambro BCT Inc., Lakewood, Colo., USA. However, it should be noted that any device known in the art for separating blood into components may be used to collect platelets without departing from the spirit and scope of the present invention.

Collected platelets were suspended in a volume of 278 mL of fluid containing a final concentration of 50 μM riboflavin and either with or without glycolytic inhibitors. 2-deoxy-D-glucose and sodium succinate are examples of glycolytic inhibitors which may be used either alone or in combination in the present invention. The platelets were irradiated in a Sengewald bag (available from Sengewald Verpackungen GmbH & Co. KG) (however any bag known in the art may be used) at 7 J/cm² with 320 nm broad band ultra violet light.

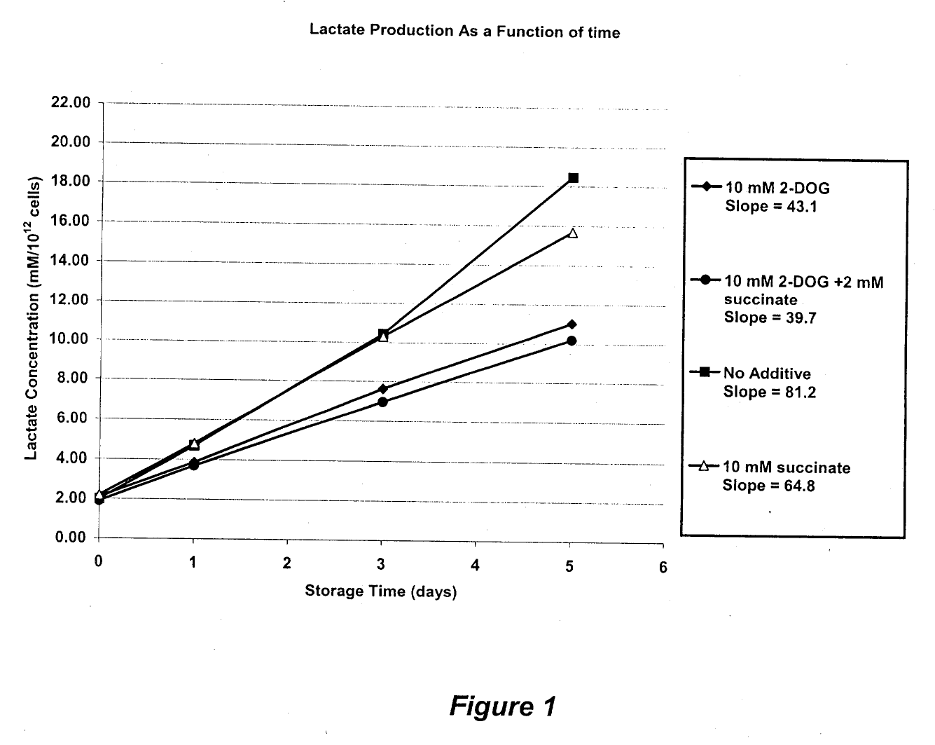

FIG. 1 shows lactate production by pathogen reduced platelets over a five day storage period. The platelets were pathogen reduced in a solution containing riboflavin and additionally may or may not have glycolytic inhibitors added. Platelets which were pathogen reduced and stored in a solution containing 10 mM 2-deoxy-D-glucose and 2 mM sodium succinate show a marked decrease in the production of lactic acid over five days in storage as compared to platelets which were pathogen reduced and stored in solution without any type of glycolytic inhibitor. This is an expected result since 2-deoxy-D-glucose blocks or slows the glycolysis pathway and consequently the breakdown of pyruvate into lactate would be significantly decreased. Sodium succinate increases H⁺ uptake in the electron transport chain and therefore should also slow the production of lactate since the stored platelets are being driven to produce energy via the citric acid cycle, not through glycolysis.

The effect of glycolytic inhibitors on the rate of lactate production by pathogen reduced platelets over time is shown more dramatically by comparing the slope of the line for each treatment. As shown in FIG. 1, platelets in a solution containing one or more glycolytic inhibitors show much lower slopes (corresponding to a slower rate of lactate production and therefore slower platelet metabolism and less platelet activation during storage) than platelets in a solution without glycolytic inhibitors. Platelets in a solution containing both 2-deoxy-D-glucose and sodium succinate have a slope of 39.7 (mM of lactate produced/10¹² cells/hr) as compared to platelets in a solution without any glycolytic inhibitors which have a slope of 81.2 (mM of lactate produced/10¹² cells/hr). Platelets in a solution containing 2-deoxy-D-glucose alone (43.1 mM of lactate produced/10¹² cells/hr) or sodium succinate alone (64.8 mM of lactate produced/10¹² cells/hr) also show lower slopes than platelets in a solution containing no additives.

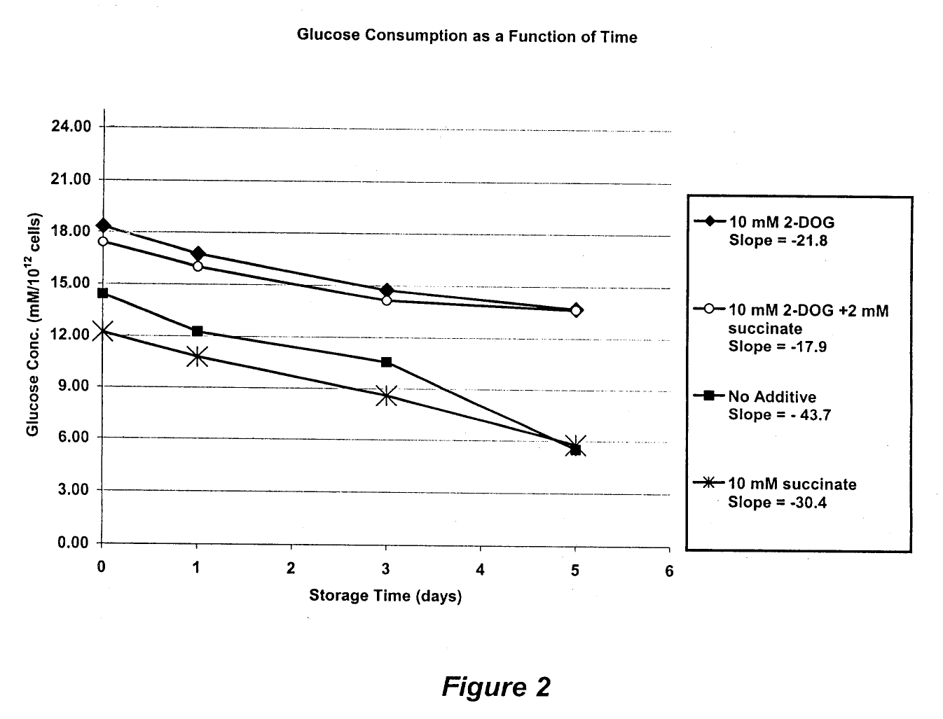

FIG. 2 shows glucose consumption of pathogen reduced platelets over time. As can be seen from FIG. 2, glucose is consumed at a slower rate by pathogen reduced platelets in a solution containing 10 mM 2-deoxy-D-glucose and 2 mM sodium succinate than by platelets containing no 2-deoxy-D-glucose or sodium succinate. This result is also expected because 2-deoxy-D-glucose inhibits or slows down the glycolytic pathway, and sodium succinate increases H⁺ uptake in the electron transport chain and therefore drives the stored platelets to produce energy via the citric acid cycle, thus the combination of both mechanisms contribute to the platelets consuming less glucose. By inhibiting or slowing down the glycolysis pathway, platelets are forced to produce ATP through the citric acid pathway, and therefore are able to consume less glucose then platelets which do not have blocked or slowed glycolysis pathways.

Again, the effect of glycolytic inhibitors on the rate of glucose consumption by pathogen reduced platelets over time is shown more dramatically by comparing the slope of the line for each treatment. As shown in FIG. 2, platelets containing one or more glycolytic inhibitors show much lower slopes (corresponding to a slower rate of glucose consumption and therefore slower platelet metabolism and less platelet activation during storage) than platelets in a solution without glycolytic inhibitors. Platelets in a solution containing both 2-deoxy-D-glucose and sodium succinate have a slope of −17.9 (mM of glucose consumed/10¹² cells/hr) as compared to platelets in a solution without any glycolytic inhibitors which have a slope of −43.7 (mM of glucose consumed/10¹² cells/hr). Platelets in a solution containg 2-deoxy-D-glucose alone (−21.8 mM of glucose consumed/10¹² cells/hr) or sodium succinate alone (−30.4 mM of glucose consumed/10¹² cells/hr) also show lower slopes than platelets in a solution containing no additives.

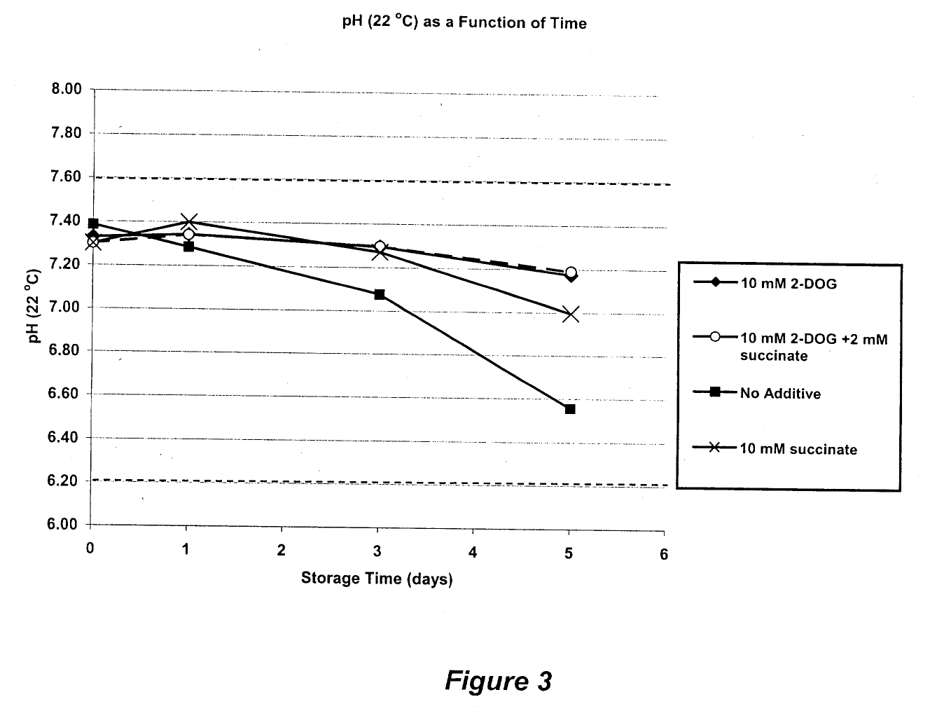

FIG. 3 shows the drop in pH over time of a solution containing pathogen reduced platelets. As shown, 10 mM 2-deoxy-D-glucose and 2 mM sodium succinate appears to maintain the pH of the platelets at approximately pH 7.40. Platelets without any glycolytic inhibitors experience a rapid drop in pH. Such results are expected because lactic acid is not produced at high rates if the glycolysis pathway is inhibited, and consequently, the pH of the solution remains at a relatively constant level.

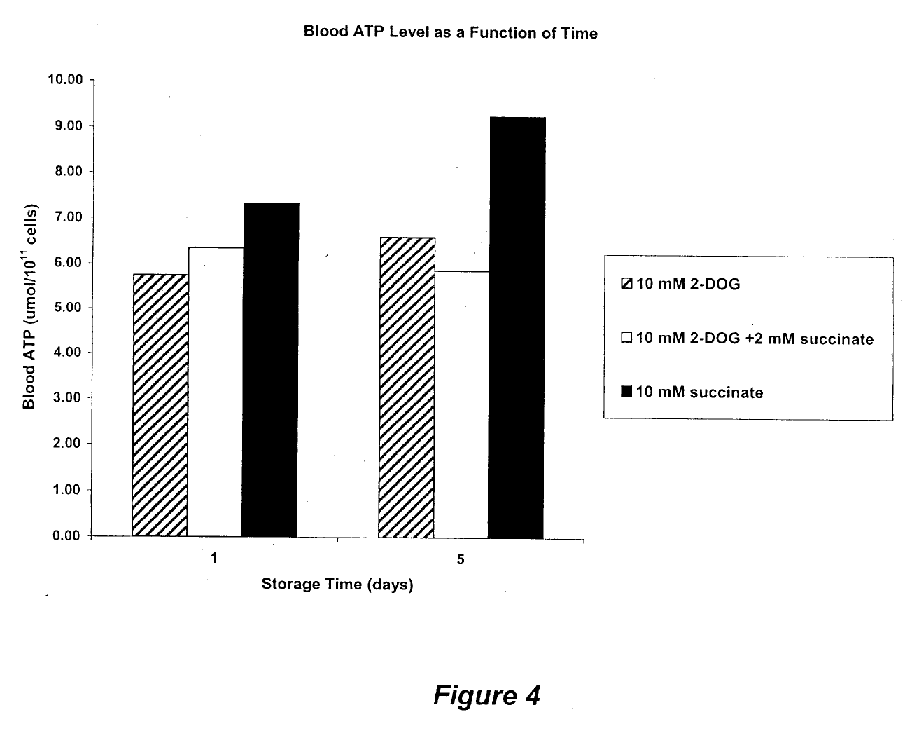

FIG. 4 is a graph showing ATP levels in pathogen reduced platelets stored over five days. ATP levels appear to remain substantially constant over five days of storage with the addition of 2-deoxy-D-glucose and sodium succinate. Sodium succinate and 2-deoxy-D-glucose alone appear to cause a slight increase in platelet ATP levels over five days of storage.

In addition to 2-deoxy-D-glucose, other sugars may find similar utility as glycolytic inhibitors. These include xylose, ribose, arabinose, and lyxose. The fact that all of these agents are natural, non-toxic and well tolerated in vivo make them ideal candidates for use in pathogen reduction and storage solutions. The above mentioned glycolytic inhibitors may be used either alone or in combination with sodium succinate.

It will be readily understood by those skilled in the art that the foregoing description has been for purposes of illustration only and that a number of changes may be made without departing from the scope of the invention. For example, photosensitizers other then those mentioned may be used, preferably photosensitizers which bind to nucleic acid and thereby keep it from replicating, and more preferably those which are not toxic and do not have toxic breakdown products. In addition, equivalent structures to those described herein for inhibiting the glycolytic pathway in platelets may be readily devised without undue experimentation by those skilled in the art following the teachings hereof.

1. A pathogen reduction solution for suspending platelets undergoing a pathogen reduction procedure comprising;
an endogenous photosensitizer;
a first glycolytic inhibitor; and
a second glycolytic inhibitor.

2. The pathogen reduction solution of claim 1 further comprising a solvent.

3. The pathogen reduction solution of claim 1 wherein the endogenous photosensitizer further comprises 7,8-dimethyl-10-ribityl isoalloxazine.

4. The pathogen reduction solution of claim 1 wherein the first glycolytic inhibitor is selected from the group consisting of 2-deoxy-D-glucose, xylose, arabinose and lyxose.

5. The pathogen reduction solution of claim 1 wherein the first glycolytic inhibitor is added at a concentration of between about 1 mM to 10 mM.

6. The pathogen reduction solution of claim 4 wherein the first glycolytic inhibitor is 2-deoxy-D-glucose at a concentration of about 10 mM.

7. The pathogen reduction solution of claim 1 wherein the second glycolytic inhibitor is added at a concentration of between about 1 mM to 10 mM.

8. The pathogen reduction solution of claim 7 wherein the second glycolytic inhibitor is sodium succinate at a concentration of about 2 mM.

9. The pathogen reduction solution of claim 2 wherein the solvent is selected from the group consisting of PSS 1, PSS 2, PSS 3, PSS 4, PSS 5, PSS 6, PSS 7, PSS 8 and PSS 9.

10. The pathogen reduction solution of claim 2 wherein the solvent is selected from the group consisting of saline and water.

11. The pathogen reduction solution of claim 1 further comprising a quencher selected from the group consisting of adenine, histidine, cysteine, tyrosine, tryptophan, ascorbate, N-acetyl-L-cysteine, propyl gallate, glutathione, mercaptopropionylglycine, dithiothreotol, nicotinamide, BHT, BHA, lysine, serine, methionine, glucose, mannitol, vitamin E, alpha-tocopherol acetate, trolox, glycerol, and mixtures thereof.

12. A storage solution for suspending platelets which have undergone a pathogen reduction procedure comprising;
an endogenous photosensitizer;
a first glycolytic inhibitor; and
a second glycolytic inhibitor.

13. The storage solution of claim 12 further comprising a solvent.

14. The storage solution of claim 12 wherein the endogenous photosensitizer further comprises 7,8-dimethyl-10-ribityl isoalloxazine.

15. The storage solution of claim 12 wherein the first glycolytic inhibitor is selected from the group consisting of 2-deoxy-D-glucose, xylose, arabinose and lyxose.

16. The storage solution of claim 12 wherein the first glycolytic inhibitor is added at a concentration of between about 1 mM to 10 mM.

17. The storage solution of claim 15 wherein the first glycolytic inhibitor is 2-deoxy-D-glucose at a concentration of about 10 mM.

18. The storage solution of claim 12 wherein the second glycolytic inhibitor is added at a concentration of between about 1 mM to 10 mM.

19. The storage solution of claim 18 wherein the second glycolytic inhibitor is sodium succinate at a concentration of about 2 mM.

20. The storage solution of claim 13 wherein the solvent is selected from the group consisting of PSS 1, PSS 2, PSS 3, PSS 4, PSS 5, PSS 6, PSS 7, PSS 8 and PSS 9.

21. The storage solution of claim 13 wherein the solvent is selected from the group consisting of saline and water.

22. The storage solution of claim 12 further comprising a quencher selected from the group consisting of adenine, histidine, cysteine, tyrosine, tryptoph an, ascorbate, N-acetyl-L-cysteine, propyl gallate, glutathione, mercaptopropionylglycine, dithiothreotol, nicotinamide, BHT, BHA, lysine, serine, methionine, glucose, mannitol, vitamin E, alpha-tocopherol acetate, trolox, glycerol, and mixtures thereof.

23. A method of producing pathogen reduced viable platelets suitable for infusing into a patient comprising:
adding an effective, non-toxic amount of an endogenous photosensitizer to the platelets to form a fluid;
adding an effective amount of a glycolytic inhibitor to the fluid; and
exposing the fluid to photoradiation sufficient to activate the photosensitizer and substantially reduce any pathogens which may be contained in the fluid.

24. The method of claim 23 wherein the viability of the platelets is maintained during the pathogen reduction procedure.

25. The method of claim 23 wherein the viability of the platelets is maintained after the pathogen reduction procedure.

26. The method of claim 23 wherein the step of adding an effective, non-toxic amount of an endogenous photosensitizer further comprises adding 7,8-dimethyl-10-ribityl isoalloxazine.

27. The method of claim 23 wherein the step of adding a glycolytic inhibitor further comprises adding a first and second glycolytic inhibitor.

28. The method of claim 27 wherein the first glycolytic inhibitor is selected from the group consisting of 2-deoxy-D-glucose, xylose, arabinose and lyxose.

29. The method of claim 27 wherein the step of adding a first glycolytic inhibitor further comprises adding the first glycolytic inhibitor at a concentration of between about 1 mM to 10 mM.

30. The method of claim 29 wherein the first glycolytic inhibitor is 2-deoxy-D-glucose at a concentration of about 10 mM.

31. The method of claim 27 wherein the step of adding a second glycolytic inhibitor further comprises adding the second glycolytic inhibitor at a concentration of between about 1 mM to 10 mM.

32. The method of claim 31 wherein the second glycolytic inhibitor is sodium succinate at a concentration of about 2 mM.

33. The method of claim 27 wherein the step of adding comprises adding to the fluid one of the first and second glycolytic inhibitors and subsequently adding the other of the first and second inhibitors.

34. The method of claim 27 wherein the step of adding comprises adding together to the fluid the first and second glycolytic inhibitors.

35. The method of claim 23 further comprising storing the platelets in the fluid resulting from the method of claim 23 to reduce any damage to the platelets that may have occurred during the pathogen reduction.
